SEQUENCE LISTING (17 sequences)

SEQ ID NO: 1 - DNA, 1863 bp, Escherichia coli
atgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataa

SEQ ID NO: 2 - protein, 620 aa, Escherichia coli
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKNRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVLGLITTLKNMRDLKGPQFLHIMTKKGRGYEPAEKDPITFHAVPKFDPSSGCLPKSSGGLPSYSKIFGDWLCETAAKDNKLMAITPAMREGSGMVEFSRKFPDRYFDVAIAEQHAVTFAAGLAIGGYKPIVAIYSTFLQRAYDQVLHDVAIQKLPVLFAIDRAGIVGADGQTHQGAFDLSYLRCIPEMVIMTPSDENECRQMLYTGYHYNDGPSAVRYPRGNAVGVELTPLEKLPIGKGIVKRRGEKLAILNFGTLMPEAAKVAESLNATLVDMRFVKPLDEALILEMAASHEALVTVEENAIMGGAGSGVNEVLMAHRKPVPVLNIGLPDFFIPQGTQEEMRAELGLDAAGMEAKIKAWLA

SEQ ID NO: 3 - DNA, 549 bp, Escherichia coli
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 4 - protein, 182 aa, Escherichia coli
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 5 - DNA, 972 bp, Escherichia coli
atgaatttagaaaaaatcaatgagttaaccgcgcaagatatggcgggtgttaatgcggcaatccttgagcagcttaattccgacgtccaactgatcaatcagttaggctattacatcgtcagcggcggcggtaaacgtattcgtccgatgattgctgtactggctgcacgagctgttggctatgagggaaatgcgcatgtcaccattgctgccctgatcgagtttatccacacggcgactctgctacacgacgacgttgtggatgaatcagatatgcgcaggggtaaagctaccgccaacgccgcatttggcaatgccgccagcgtgctggtaggcgattttatttatacccgcgctttccagatgatgaccagcctcggttcgctcaaagtgctggaagtcatgtcagaagccgtaaacgtcatcgcagaaggtgaagttctgcaactgatgaacgttaacgatccggacatcactgaagaaaactacatgcgcgttatctatagcaaaaccgcgcgtctgtttgaggctgccgcgcagtgttccgggattctggctggctgtacgccggaggaggagaaaggcctgcaggattatgggcgctatctcggcactgctttccagttgatcgacgatttactcgattacaatgccgatggcgaacagttaggtaaaaatgtcggcgacgatctgaacgaaggtaaaccgacgctgccgctgctgcatgcgatgcatcatggcacaccagaacaggcacagatgatccgtaccgccatcgaacagggtaacggtcgccatcttctggaaccggttctggaagcaatgaacgcttgtggatctcttgaatggacgcgtcagcgtgccgaggaagaagcagacaaagccatcgcagcgttacaggtgctcccggacaccccttggcgagaagcactcatcggcctcgcgcacatcgctgttcaacgcgatcgttaa

SEQ ID NO: 6 - protein, 323 aa, Escherichia coli
MNLEKINELTAQDMAGVNAAILEQLNSDVQLINQLGYYIVSGGGKRIRPMIAVLAARAVGYEGNAHVTIAALIEFIHTATLLHDDVVDESDMRRGKATANAAFGNAASVLVGDFIYTRAFQMMTSLGSLKVLEVMSEAVNVIAEGEVLQLMNVNDPDITEENYMRVIYSKTARLFEAAAQCSGILAGCTPEEEKGLQDYGRYLGTAFQLIDDLLDYNADGEQLGKNVGDDLNEGKPTLPLLHAMHHGTPEQAQMIRTAIEQGNGRHLLEPVLEAMNACGSLEWTRQRAEEEADKAIAALQVLPDTPWREALIGLAHIAVQRDR

SEQ ID NO: 7 - DNA, 711 bp, Escherichia coli
atggcaaccactcatttggatgtttgcgccgtggttccggcggccggatttggccgtcgaatgcaaacggaatgtcctaagcaatatctctcaatcggtaatcaaaccattcttgaacactcggtgcatgcgctgctggcgcatccccgggtgaaacgtgtcgtcattgccataagtcctggcgatagccgttttgcacaacttcctctggcgaatcatccgcaaatcaccgttgtagatggcggtgatgagcgtgccgattccgtgctggcaggtctgaaagccgctggcgacgcgcagtgggtattggtgcatgacgccgctcgtccttgtttgcatcaggatgacctcgcgcgattgttggcgttgagcgaaaccagccgcacgggggggatcctcgccgcaccagtgcgcgatactatgaaacgtgccgaaccgggcaaaaatgccattgctcataccgttgatcgcaacggcttatggcacgcgctgacgccgcaatttttccctcgtgagctgttacatgactgtctgacgcgcgctctaaatgaaggcgcgactattaccgacgaagcctcggcgctggaatattgcggattccatcctcagttggtcgaaggccgtgcggataacattaaagtcacgcgcccggaagatttggcactggccgagttttacctcacccgaaccatccatcaggagaatacataa

SEQ ID NO: 8 - protein, 236 aa, Escherichia coli
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLTRTIHQENT

SEQ ID NO: 9 - DNA, 480 bp, Escherichia coli
atgcgaattggacacggttttgacgtacatgcctttggcggtgaaggcccaattatcattggtggcgtacgcattccttacgaaaaaggattgctggcgcattctgatggcgacgtggcgctccatgcgttgaccgatgcattgcttggcgcggcggcgctgggggatatcggcaagctgttcccggataccgatccggcatttaaaggtgccgatagccgcgagctgctacgcgaagcctggcgtcgtattcaggcgaagggttatacccttggcaacgtcgatgtcactatcatcgctcaggcaccgaagatgttgccgcacattccacaaatgcgcgtgtttattgccgaagatctcggctgccatatggatgatgttaacgtgaaagccactactacggaaaaactgggatttaccggacgtggggaagggattgcctgtgaagcggtggcgctactcattaaggcaacaaaatga

SEQ ID NO: 10 - protein, 159 aa, Escherichia coli
MRIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 11 - DNA, 924 bp, Pantoea agglomerans
atggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctga

SEQ ID NO: 12 - protein, 307 aa, Pantoea agglomerans
MVSGSKAGVSPHREIEVMRQSIDDHLAGLLPETDSQDIVSLAMREGVMAPGKRIRPLLMLLAARDLRYQGSMPTLLDLACAVELTHTASLMLDDMPCMDNAELRRGQPTTHKKFGESVAILASVGLLSKAFGLIAATGDLPGERRAQAVNELSTAVGVQGLVLGQFRDLNDAALDRTPDAILSTNHLKTGILFSAMLQIVAIASASSPSTRETLHAFALDFGQAFQLLDDLRDDHPETGKDRNKDAGKSTLVNRLGADAARQKLREHIDSADKHLTFACPQGGAIRQFMHLWFGHHLADWSPVMKIA

SEQ ID NO: 13 - DNA, 2409 bp, Taxus baccata
atgagcagtagcactggcactagcaaggtggtttccgagacttccagtaccattgtggatgatatccctcgactctccgccaattatcatggcgatctgtggcaccacaatgttatacaaactctggagacaccatttcgtgagagttctactttccaagaacgggcagacgagctggttgtgaaaattaaagatatgttcaatgcgctcggagacggagatatcagtccgtctgcatacgacactgcgtgggtggcgagggtggcgaccgtttcctctgatggatctgagaagccacggtttcctcaggccctcaactgggttttaaacaaccagctccaagatggatcatggggtatcgaatcgcactttagtttatgcgatcgattgcttaacacggtcaattctgttatcgccctctcggtttggaaaacagggcacagccaagtagaacaaggtactgagtttattgcagagaatctaagattactcaatgaggaagatgagttgtccccggatttcgaaataatctttcctgctctgctgcaaaaggcaaaagcgttggggatcaatcttccttacgatcttccatttatcaaatctttgtcgacaacacgggaagccaggcttacagatgtttctgcggcagcagacaatattccagccaacatgttgaatgcgttggagggtctggaggaagttattgattggaacaagattatgaggtttcaaagtaaagatggatctttcctgagctcccctgcctccactgcctgtgtactgatgaatacaggggacgaaaaatgtttcactcttctcaacaatctgctggacaaattcggcggctgcgtgccctgtatgtattccatcgatctgctggaacgcctttcgctggttgataacattgagcatctcggaatcggtcgccatttcaaacaagaaatcaaagtagctcttgattatgtctacagacattggagtgaaaggggcatcggttggggcagagacagccttgttccagatctcaacacaacagccctcggcctgcgaactcttcgcacgcacggatacgatgtttcttcagatgttttgaataatttcaaagatgaaaacgggcggttcttctcctctgcgggccaaacccatgtcgaattgagaagcgtggtgaatcttttcagagcttccgaccttgcatttcctgacgaaggagctatggacgatgctagaaaatttgcagaaccatatcttagagacgcacttgcaacgaaaatctcaaccaatacaaaactatacaaagagattgagtacgtggtggagtacccttggcacatgagtatcccacgcctagaagctagaagttatattgattcgtatgacgacgattatgtatggcagaggaagactctatacagaatgccatctttgagtaattcaaaatgtttagaattggcaaaattggacttcaatatcgtacaatctttgcatcaagaggagttgaagcttctaacaagatggtggaaggaatccggcatggcagatataaatttcactcgacaccgagtggcggaggtttatttttcatcagctacatttgaacctgaatattctgccaccagaattgccttcacaaaaattggttgtttacaagtcctttttgatgatatggctgacatctttgcaacactagatgaattgaaaagtttcactgagggagtaaagagatgggatacatctttgctacatgagattccagagtgtatgcaaacttgctttaaagtttggttcaaattaatggaagaagtaaataatgatgtggttaaggtacaaggacgtgacatgctcgctcacataagaaaaccttgggagttgtacttcaattgttacgtacaagaaagggagtggcttgaagctgggtatataccaacttttgaagagtacttaaagacttatgctatatcagtaggccttggaccgtgtaccctacaaccaatactactgatgggtgagcttgtgaaagatgatgttgttgagaaagtgcactatccctcaaatatgtttgagcttgtatccttgagctggcgactaacaaacgacaccaaaacatatcaggctgaaaaggctcgaggacaacaagcctcaggcatagcatgctatatgaaggataatccaggagcaactgaggaagatgccatcaagcacatatgtcgtgttgttgaccgggccttgaaagaagcaagctttgaatatttcaaaccatccaatgatatcccaatgggttgcaagtcctttatttttaaccttagattgtgtgtccaaatattttacaagtttatagatgggtacggaatcgccaatgaggagattaaggattatataagaaaagtttatattgatccaattcaagtatga

SEQ ID NO: 14 - protein, 802 aa, Taxus baccata
MSSSTGTSKVVSETSSTIVDDIPRLSANYHGDLWHHNVIQTLETPFRESSTFQERADELVVKIKDMFNALGDGDISPSAYDTAWVARVATVSSDGSEKPRFPQALNWVLNNQLQDGSWGIESHFSLCDRLLNTVNSVIALSVWKTGHSQVEQGTEFIAENLRLLNEEDELSPDFEIIFPALLQKAKALGINLPYDLPFIKSLSTTREARLTDVSAAADNIPANMLNALEGLEEVIDWNKIMRFQSKDGSFLSSPASTACVLMNTGDEKCFTLLNNLLDKFGGCVPCMYSIDLLERLSLVDNIEHLGIGRHFKQEIKVALDYVYRHWSERGIGWGRDSLVPDLNTTALGLRTLRTHGYDVSSDVLNNFKDENGRFFSSAGQTHVELRSVVNLFRASDLAFPDEGAMDDARKFAEPYLRDALATKISTNTKLYKEIEYVVEYPWHMSIPRLEARSYIDSYDDDYVWQRKTLYRMPSLSNSKCLELAKLDFNIVQSLHQEELKLLTRWWKESGMADINFTRHRVAEVYFSSATFEPEYSATRIAFTKIGCLQVLFDDMADIFATLDELKSFTEGVKRWDTSLLHEIPECMQTCFKVWFKLMEEVNNDVVKVQGRDMLAHIRKPWELYFNCYVQEREWLEAGYIPTFEEYLKTYAISVGLGPCTLQPILLMGELVKDDVVEKVHYPSNMFELVSLSWRLTNDTKTYQAEKARGQQASGIACYMKDNPGATEEDAIKHICRVVDRALKEASFEYFKPSNDIPMGCKSFIFNLRLCVQIFYKFIDGYGIANEEIKDYIRKVYIDPIQV

SEQ ID NO: 15 - DNA, 891 bp, Artificial Sequence
  T. cuspidata crtE gene optimized for codon usage in E. coli
atgttcgacttcaacgagtacatgaaatcgaaagcagttgcagttgatgctgcgcttgacaaagcgattccgctggaataccctgaaaagattcacgaatcgatgcgttatagtctgctggctggtggcaaacgcgtgcgcccagctctttgcattgcggcatgtgagctggtaggcggttcccaggatctggctatgccaacggcgtgcgcaatggaaatgatccatacaatgtccctgatccacgatgatctgccgtgtatggataatgatgacttccgccgtggaaaaccgactaaccataaagtatttggcgaggacactgcagtgttggcaggagacgccctgttgagctttgcctttgaacatattgccgtcgcgacctcaaaaacagttccttctgatcgtaccctgcgcgtcatcagtgagttaggtaagaccattggcagccaggggctggtaggcggccaggtcgtggatatcacgtctgaaggtgacgcgaatgtggatcttaagaccttagagtggattcacatccataaaacggccgtgctgctggaatgctcggttgtgtccggtgggatcctggggggcgccactgaggacgaaatcgcccgtattcgccgttatgcacggtgtgtgggcctcttgtttcaagtcgtggatgatattctggatgtgacgaaatctagtgaggagctcggtaaaaccgcgggcaaggatctcctgaccgacaaggcgacgtacccgaaactgatgggtttggaaaaggctaaggagtttgctgccgaattagcgaccagagccaaagaagaactctcttctttcgaccagatcaaggcagcgccccttttagggctggcggattatattgcctttcgtcaaaactaa

SEQ ID NO: 16 - protein, 296 aa, Taxus cuspidata
MFDFNEYMKSKAVAVDAALDKAIPLEYPEKIHESMRYSLLAGGKRVRPALCIAACELVGGSQDLAMPTACAMEMIHTMSLIHDDLPCMDNDDFRRGKPTNHKVFGEDTAVLAGDALLSFAFEHIAVATSKTVPSDRTLRVISELGKTIGSQGLVGGQVVDITSEGDANVDLKTLEWIHIHKTAVLLECSVVSGGILGGATEDEIARIRRYARCVGLLFQVVDDILDVTKSSEELGKTAGKDLLTDKATYPKLMGLEKAKEFAAELATRAKEELSSFDQIKAAPLLGLADYIAFRQN

SEQ ID NO: 17 - DNA, 2415 bp, Artificial Sequence
  T. baccata txs gene optimized for codon usage in E. coli
catatgatgtctagctctacgggtacgtctaaagtcgtgagtgaaacctcatcgacgatcgtggacgatattccacgcttgtcggcgaactatcatggagatctgtggcatcataacgtcattcagacattggaaaccccgtttcgcgaaagtagcacctaccaggaacgggcagatgaattagtcgtgaaaatcaaagatatgtttaatgcattaggagatggagacatctcgcccagcgcatatgatacggcgtgggtggctcggttggccacgattagctccgatggcagtgaaaagccgcgtttcccgcaggcgctgaactgggtgtttaataatcaattgcaggatggcagctggggcattgaatctcactttagcctctgtgaccggttactcaacacgacaaactccgtaattgcgttgtcagtttggaaaacgggccatagccaggttcaacagggcgcggaatttatcgctgaaaatctgcgcctgctgaacgaggaggacgaactgtcacccgattttcagattatttttccggctttactccagaaagccaaagccttaggcatcaacctgccatatgatctgccgttcatcaagtatctgtctactacccgcgaagcccgtctcactgacgtctctgcggcggcggacaatattccagcgaacatgctgaacgcactggaagggctggaagaggttatcgactggaataaaatcatgcgcttccaaagcaaggacggtagcttcttaagcagcccagcatctactgcttgtgttctgatgaataccggagacgaaaagtgctttacgtttctgaacaatctgctggacaaatttgggggttgtgttccttgtatgtattccattgatctgttggaacgtctgtcgctggtcgataacattgaacacttaggtatcggccgccacttcaaacaagaaatcaagggggcgttggattatgtataccgtcattggagcgagcgtggtattggttgggggcgcgatagcttggtacctgatctgaacaccactgctttgggactgcgcactcttcgtatgcacggatacaacgttagttccgatgtcctcaataatttcaaggacgagaacggccgttttttcagctcggccggtcagacgcatgttgaactgcggtccgtagtcaatctctttcgcgctagtgatctggccttccccgacgagcgcgctatggacgatgcacggaagtttgccgagccgtatctccgcgaagccctggccaccaaaatttcaaccaacaccaagcttttcaaagaaattgagtatgtagtagagtatccgtggcatatgtctattccgcgcctggaagcccgctcgtatatcgattcttacgatgacaattatgtgtggcaacgcaaaacactgtaccgtatgcccagcctgtcaaatagtaagtgtctggagctggcgaaactggatttcaacattgtgcaatccctgcaccaagaagagctgaaattactgactcgctggtggaaggaatccggcatggcagacatcaattttacgcgtcaccgtgttgcagaggtgtacttctcctcggcgacctttgagccggagtattcggccacacgtattgcatttaccaagattggctgccttcaggtgctttttgacgatatggcggatatttttgcgacacttgatgagcttaaatcatttaccgaaggcgtgaagcgttgggatacctctctgttgcatgaaatccccgaatgtatgcagacctgcttcaaagtttggttcaaactgatggaagaagtgaacaacgacgtcgtgaaagttcagggtcgtgatatgttagcacacatccgcaagccgtgggaactctatttcaattgctatgtgcaggagcgtgaatggttagaagcgggctacattcctaccttcgaagagtacttaaaaacctatgccatttccgtcggtttaggcccgtgcactctgcagcctatcttgctgatgggtgagctggtaaaggatgatgtggtggaaaaagttcactacccgtcgaatatgtttgaactggtaagtctgagttggcgtctgacaaacgacaccaaaacgtaccaggcagaaaaggcacgtgggcaacaggcaagcggtatcgcgtgttatatgaaggataatccgggcgctactgaggaagatgccattaagcatatctgccgtgttgtggatcgcgctcttaaagaagcgtcattcgaatattttaaacctagtaatgatattccgatgggttgtaagtcattcattttcaatcttcgcctgtgcgtgcaaattttttacaaatttattgacggctacggaatcgccaacgaagaaatcaaagactatattcgtaaagtttacatcgatccaatccaggtctaa

(19) United States
(12) Patent Application Publication
(10) Pub. No.: US 2010/0261168 A1
(43) Pub. Date: Oct. 14, 2010

(54) Screening for Environmental DNAs Encoding Enzymes for Synthesizing Terpenoid-Based Therapeutic Compounds Using Genetically Modified E. Coli Strains

(75) Inventors: Blaine Pfeifer, Somerville, MA (US); Yong Wang, Shanghai (CN)

Correspondence Address: OCCHIUTI ROHLICEK & TSAO, LLP, 10 FAWCETT STREET, CAMBRIDGE, MA, 02138, US

(73) Assignee: Tufts University

(21) Appl. No.: 12/420,502

(22) Filed: Apr. 8, 2009

(51) Int. Cl. C12Q 1/68

(52) U.S. Cl. 435/6

(57) ABSTRACT

A screening method for identifying microbial genes involved in biosynthesis of therapeutic terpenoid-based compounds using genetically modified E. coli strains that yield high levels of products of the non-mevalonate 1-deoxy-D-xylulose-5-phosphate pathway.

### STATEMENT AS TO FEDERALLY SPONSORED RESEARCH

This invention was made with government support under 0712019 awarded by National Science Foundation. The government has certain rights in the invention.

### BACKGROUND OF THE INVENTION

Many environmental microbes produce therapeutically effective terpenoid-based compounds, i.e., compounds synthesized from terpenoid compounds. They carry genes encoding enzymes that convert terpenoid compounds, i.e., products of the non-mevalonate 1-deoxy-D-xylulose-5-phosphate (DXP) pathway, to terpenoid-based compounds of interest. In general, it is not feasible to directly isolate these genes from the microbes, most of which cannot be cultured.

To isolate these genes of interest, one possible way is to introduce DNAs of environmental microbes into a culturable host and cells of the host are then screened for those that secret therapeutically effective terpenoid-based compounds. While E. coli is an ideal host, native E. coli strains produce low levels of terpenoid compounds to be converted to therapeutic terpenoid-based compounds due to poor expression of the genes involved in the DXP pathway.

### SUMMARY OF THE INVENTION

The present invention features a method of screening for microbial DNAs that encode an enzyme or enzymes involved in the synthesis of a therapeutically effective terpenoid-based compound using a genetically modified E. coli strain that expresses high levels of terpenoid compounds, the products of the DXP pathway.

In one example, this method includes the following steps: (1) transforming the genetically modified E. coli strain with an expression plasmid containing a microbial DNA to obtain a transformant, (2) contacting substances released from the transformant with a tester microorganism suspected of being sensitive to a terpenoid-based antibiotic, and (3) determining whether or not the substances inhibits growth of the tester microorganism. Growth inhibition indicates that the microbial DNA encodes an enzyme or enzymes for synthesis of a terpenoid-based antibiotic (e.g., an anti-bacterial or anti-fungal compound). The contacting step can be performed by cultivating the transformant in a culturing medium to allow expression of the microbial DNA, collecting the culturing medium after cultivation, and contacting the culturing medium with the tester microorganism. Alternatively, this step is performed by cultivating the transformant to form a colony on an agar plate and contacting the colony with a lawn of the tester microorganism.

The genetically modified E. coli strain used in the method described above overexpresses one or more genes involved in the DXP pathway, e.g., genes of dxs (encoding 1-deoxyxylulose-5-phosphate synthase), idi (encoding isopentenyl diphosphate isomerase), ispB (encoding octaprenyl diphosphate synthase), ispD (encoding 4-diphosphocytidyl-2C-methyl-D-erythritol synthase), and ispF (encoding 2-C-methyl-Derythritol 2,4-cyclodiphosphate synthase). More specifically, this E. coli strain contains an exogenous expression cassette containing dxs and idi genes and, optionally, ispB, ispD, or ispF genes, all of which are under the control of an E. coli promoter, e.g., pCold, pLac, SP6, T3, T5, T7, Tac, or Trc. The exogenous expression cassette can be integrated into the E. coli chromosome, e.g., at the araA locus. Preferably, the E. coli strain has non-functional gdhA, aceA, or fdhF genes.

In another example, the method of this invention includes (1) transforming the genetically modified E. coli strain, described above, with an expression plasmid carrying a microbial DNA to obtain a transformant, (2) cultivating the transformant in a culturing medium to allow expression of the microbial DNA, (3) collecting the culturing medium after cultivation, (4) contacting the culturing medium with cancer cells, and (5) determining whether or not the culturing medium exerts a cytotoxic effect on the cancer cells. Cytotoxic effect indicates that the microbial DNA encodes an enzyme or enzymes for synthesis of an anti-cancer terpenoid-based compound.

The details of one or more embodiments of the invention are set forth in the description below. Other features or advantages of the present invention will be apparent from the detailed description of several examples and also from the appending claims.

### DETAILED DESCRIPTION OF THE INVENTION

Disclosed herein is use of a genetically modified E. coli strain to screen for microbial DNAs that encode an enzyme or enzymes involved in the synthesis of a therapeutically effective terpenoid-based compound, i.e., an antibiotic, anticancer, antifungal, or immunosuppressant agent.

The genetically modified E. coli strain contains an exogenous expression cassette that includes an E. coli promoter operably linked to one or more of the genes involved in the non-mevalonate 1-deoxy-D-xylulose-5-phosphate (DXP) biosynthetic pathway (“DXP genes”).

An expression cassette is an artificial nucleotide sequence including a promoter sequence operably linked to one or more coding sequences. A promoter sequence is a nucleotide sequence containing elements that initiates the transcription of an operably linked nucleic acid sequence. At a minimum, a promoter contains an RNA polymerase binding site. It can further contain one or more enhancer elements which, by definition, enhances transcription, or one or more regulatory elements that control the on/off status of the promoter. An E. coli promoter is a promoter that functions within E. coli. Representative E. coli promoters include the β-lactamase and lactose promoter systems (see Chang et al., Nature 275:615-624, 1978), the SP6, T3, T5, and T7 RNA polymerase promoters (Studier et al., Meth. Enzymol. 185:60-89, 1990), the lambda promoter (Elvin et al., Gene 87:123-126, 1990), the trp promoter (Nichols and Yanofsky, Meth. in Enzymology 101:155-164, 1983), the Tac and Trc promoters (Russell et al., Gene 20:231-243, 1982), and pCold (described in U.S. Pat. No. 6,479,260).

Any of the E. coli promoters mentioned above can be operably linked to one or more of the disclosed genes via conventional recombinant technology to construct an exogenous expression cassette. When more than one gene is under the control of the same promoter, a ribosomal binding site (RBS) can be inserted adjacent to the 5′ end of each of the genes to facilitate the translation of each gene.

DXP genes, including idi, dxs, ispA, ispB, ispC, ispD, ispF, ispG, and ispH have been identified in various species, e.g., E. coli, S. dysenteriae, Y. pestis, V. cholerae, and P. holoplanktis. Table 1 below lists examples of these genes, the corresponding species from which they derive, and the GenBank accession numbers of the encoded proteins:

TABLE 1
Exemplary DXP genes

| Gene | Species | | | | | | |
|---|---|---|---|---|---|---|---|
| dxs | E. coli | S. sonnei | S. enterica | Y. pestis | V. cholerae | H. influenzae | |
| | NP_414954 | YP_309408 | ZP_02830967 | NP_406652 | ZP_01681374 | ZP_01788524 | |
| | GI: 16128405 | GI: 74310989 | GI: 168818967 | GI: 16123339 | GI: 121728344 | GI: 145632791 | |
| | (May 17, 2008) | (Jul. 24, 2008) | (Sep. 19, 2008) | (Jul. 20, 2008) | (Jan. 11, 2007) | (Apr. 23, 2007) | |
| idi | E. coli | S. dysenteriae | S. enterica | K. pneumoniae | C. diphtheriae | M. tuberculosis | |
| | NP_417365 | YP_404694 | ZP_02835070 | YP_002236654 | NP_940068 | NP_216261 | |
| | GI: 16130791 | GI: 82778345 | GI: 168823070 | GI: 206579322 | GI: 38234301 | GI: 1560883 | |
| | (May 17, 2008) | (Jul. 24, 2008) | (Sep. 10, 2008) | (Sep. 24, 2008) | (Jul. 21, 2008) | (Jul. 18, 2008) | |
| ispB | E. coli | S. dysenteriae | S. enterica | K. pneumoniae | Y. pestis | V. cholerae | P. haloplanktis |
| | NP_417654 | YP_404850 | NP_457684 | YP_002236401 | NP_406972 | YP_002067102 | YP_341147 |
| | GI: 16131077 | GI: 82778501 | GI: 16762067 | GI: 206578772 | GI: 16123659 | GI: 194531655 | GI: 77361572 |
| | (May 17, 2008) | (Jul. 24, 2008) | (Sep. 23, 2008) | (Sep. 24, 2008) | (Jul. 20, 2008) | (Aug. 5, 2008) | (Sep. 30, 2008) |
| ispD | E. coli | S. dysenteriae | S. enterica | K. pneumoniae | Y. pestis | V. cholerae | H. influenzae |
| | NP_417227 | ZP_03064012 | YP_217849 | YP_001336740 | NP_406824 | YP_001216029 | NP_438832 |
| | GI: 16130654 | GI: 194431721 | GI: 62181432 | GI: 152971631 | GI: 16123511 | GI: 147673316 | GI: 16272614 |
| | (5/17/0808) | (Jul. 24, 2008) | (Sep. 23, 2008) | (Jul. 22, 2008) | (Jul. 20, 2008) | (Jul. 25, 2008) | (Jul. 18, 2008) |
| ispF | E. coli | S. enterica | K. pneumoniae | Y. pestis | H. influenzae | S. putrefaciens | |
| | NP_289295 | ZP_02574264 | YP_001336739 | NP_406823 | NP_438831 | ZP_01705665 | |
| | GI: 15803263 | GI: 167993169 | GI: 152971630 | GI: 16123510 | GI: 16272613 | GI: 124546598 | |
| | (Jul. 18, 2008) | (Sep. 19, 2008) | (Jul. 22, 2008) | (Jul. 20, 2008) | (Jul. 18, 2008) | (2/707) | |

In one example, one or more of E. coli DXP genes are used to construct the exogenous expression cassette described above. The nucleotide sequences of some of these genes and their encoded amino acid sequences are shown below:

```
Nucleotide sequence (SEQ ID NO: 1) of E. coli dxs gene and
the encoded amino acid sequence (SEQ ID NO: 2)
atgagttttgatattgccaaatacccgaccctggcactggtcgactccacccaggagtta
  M S F D I A K Y P T L A L V D S T Q E L
cgactgttgccgaaagagagtttaccgaaactctgcgacgaactgcgccgctatttactc
  R L L P K E S L P K L C D E L R R Y L L
gacagcgtgagccgttccagcgggcacttcgcctccgggctgggcacggtcgaactgacc
  D S V S R S S G H F A S G L G T V E L T
gtggcgctgcactatgtctacaacaccccgtttgaccaattgatttgggatgtggggcat
  V A L H Y V Y N T P F D Q L I W D V G H
caggcttatccgcataaaattttgaccggacgccgcgacaaaatcggcaccatccgtcag
  Q A Y P H K I L T G R R D K I G T I R Q
aaaggcggtctgcacccgttcccgtggcgcggcgaaagcgaatatgacgtattaagcgtc
  K G G L H P F P W R G E S E Y D V L S V
gggcattcatcaacctccatcagtgccggaattggtattgcggttgctgccgaaaaagaa
  G H S S T S I S A G I G I A V A A E K E
ggcaaaaatcgccgcaccgtctgtgtcattggcgatggcgcgattaccgcaggcatggcg
  G K N R R T V C V I G D G A I T A G M A
tttgaagcgatgaatcacgcgggcgatatccgtcctgatatgctggtgattctcaacgac
  F E A M N H A G D I R P D M L V I L N D
aatgaaatgtcgatttccgaaaatgtcggcgcgctcaacaaccatctggcacagctgctt
  N E M S I S E N V G A L N N H L A Q L L
tccggtaagctttactcttcactgcgcgaaggcgggaaaaaagttttctctggcgtgccg
  S G K L Y S S L R E G G K K V F S G V P
ccaattaaagagctgctcaaacgcaccgaagaacatattaaaggcatggtagtgcctggc
  P I K E L L K R T E E H I K G M V V P G
acgttgtttgaagagctgggctttaactacatcggcccggtggacggtcacgatgtgctg
  T L F E E L G F N Y I G P V D G H D V L
gggcttatcaccacgctaaagaacatgcgcgacctgaaaggcccgcagttcctgcatatc
  G L I T T L K N M R D L K G P Q F L H I
atgaccaaaaaaggtcgtggttatgaaccggcagaaaaagacccgatcactttccacgcc
  M T K K G R G Y E P A E K D P I T F H A
gtgcctaaatttgatccctccagcggttgtttgccgaaaagtagcggcggtttgccgagc
  V P K F D P S S G C L P K S S G G L P S
tattcaaaaatctttggcgactggttgtgcgaaacggcagcgaaagacaacaagctgatg
  Y S K I F G D W L C E T A A K D N K L M
gcgattactccggcgatgcgtgaaggttccggcatggtcgagttttcacgtaaattcccg
  A I T P A M R E G S G M V E F S R K F P
gatcgctacttcgacgtggcaattgccgagcaacacgcggtgacctttgctgcgggtctg
  D R Y F D V A I A E Q H A V T F A A G L
gcgattggtgggtacaaacccattgtcgcgatttactccactttcctgcaacgcgcctat
  A I G G Y K P I V A I Y S T F L Q R A Y
gatcaggtgctgcatgacgtggcgattcaaaagcttccggtcctgttcgccatcgaccgc
  D Q V L H D V A I Q K L P V L F A I D R
gcgggcattgttggtgctgacggtcaaacccatcagggtgcttttgatctctcttacctg
  A G I V G A D G Q T H Q G A F D L S Y L
cgctgcataccggaaatggtcattatgaccccgagcgatgaaaacgaatgtcgccagatg
  R C I P E M V I M T P S D E N E C R Q M
ctctataccggctatcactataacgatggcccgtcagcggtgcgctacccgcgtggcaac
  L Y T G Y H Y N D G P S A V R Y P R G N
gcggtcggcgtggaactgacgccgctggaaaaactaccaattggcaaaggcattgtgaag
  A V G V E L T P L E K L P I G K G I V K
cgtcgtggcgagaaactggcgatccttaactttggtacgctgatgccagaagcggcgaaa
  R R G E K L A I L N F G T L M P E A A K
gtcgccgaatcgctgaacgccacgctggtcgatatgcgttttgtgaaaccgcttgatgaa
  V A E S L N A T L V D M R F V K P L D E
gcgttaattctggaaatggccgccagccatgaagcgctggtcaccgtagaagaaaacgcc
  A L I L E M A A S H E A L V T V E E N A
attatgggcggcgcaggcagcggcgtgaacgaagtgctgatggcccatcgtaaaccagta
  I M G G A G S G V N E V L M A H R K P V
cccgtgctgaacattggcctgccggacttctttattccgcaaggaactcaggaagaaatg
  P V L N I G L P D F F I P Q G T Q E E M
cgcgccgaactcggcctcgatgccgctggtatggaagccaaaatcaaggcctggctggca
  R A E L G L D A A G M E A K I K A W L A
taa
Nucleotide sequence (SEQ ID NO: 3) of E. coli idi gene and
the encoded amino acid sequence (SEQ ID NO: 4)
atgcaaacggaacacgtcattttattgaatgcacagggagttcccacgggtacgctggaa
  M Q T E H V I L L N A Q G V P T G T L E
aagtatgccgcacacacggcagacacccgcttacatctcgcgttctccagttggctgttt
  K Y A A H T A D T R L H L A F S S W L F
aatgccaaaggacaattattagttacccgccgcgcactgagcaaaaaagcatggcctggc
  N A K G Q L L V T R R A L S K K A W P G
gtgtggactaactcggtttgtgggcacccacaactgggagaaagcaacgaagacgcagtg
  V W T N S V C G H P Q L G E S N E D A V
atccgccgttgccgttatgagcttggcgtggaaattacgcctcctgaatctatctatcct
  I R R C R Y E L G V E I T P P E S I Y P
gactttcgctaccgcgccaccgatccgagtggcattgtggaaaatgaagtgtgtccggta
  D F R Y R A T D P S G I V E N E V C P V
tttgccgcacgcaccactagtgcgttacagatcaatgatgatgaagtgatggattatcaa
  F A A R T T S A L Q I N D D E V M D Y Q
tggtgtgatttagcagatgtattacacggtattgatgccacgccgtgggcgttcagtccg
  W C D L A D V L H G I D A T P W A F S P
tggatggtgatgcaggcgacaaatcgcgaagccagaaaacgattatctgcatttacccag
  W M V M Q A T N R E A R K R L S A F T Q
cttaaataa
  L K -
Nucleotide sequence (SEQ ID NO: 5) of E. coli ispB gene and
the encoded amino acid sequence (SEQ ID NO: 6)
atgaatttagaaaaaatcaatgagttaaccgcgcaagatatggcgggtgttaatgcggca
  M N L E K I N E L T A Q D M A G V N A A
atccttgagcagcttaattccgacgtccaactgatcaatcagttaggctattacatcgtc
  I L E Q L N S D V Q L I N Q L G Y Y I V
agcggcggcggtaaacgtattcgtccgatgattgctgtactggctgcacgagctgttggc
  S G G G K R I R P M I A V L A A R A V G
tatgagggaaatgcgcatgtcaccattgctgccctgatcgagtttatccacacggcgact
  Y E G N A H V T I A A L I E F I H T A T
ctgctacacgacgacgttgtggatgaatcagatatgcgcaggggtaaagctaccgccaac
  L L H D D V V D E S D M R R G K A T A N
gccgcatttggcaatgccgccagcgtgctggtaggcgattttatttatacccgcgctttc
  A A F G N A A S V L V G D F I Y T R A F
cagatgatgaccagcctcggttcgctcaaagtgctggaagtcatgtcagaagccgtaaac
  Q M M T S L G S L K V L E V M S E A V N
gtcatcgcagaaggtgaagttctgcaactgatgaacgttaacgatccggacatcactgaa
  V I A E G E V L Q L M N V N D P D I T E
gaaaactacatgcgcgttatctatagcaaaaccgcgcgtctgtttgaggctgccgcgcag
  E N Y M R V I Y S K T A R L F E A A A Q
tgttccgggattctggctggctgtacgccggaggaggagaaaggcctgcaggattatggg
  C S G I L A G C T P E E E K G L Q D Y G
cgctatctcggcactgctttccagttgatcgacgatttactcgattacaatgccgatggc
  R Y L G T A F Q L I D D L L D Y N A D G
gaacagttaggtaaaaatgtcggcgacgatctgaacgaaggtaaaccgacgctgccgctg
  E Q L G K N V G D D L N E G K P T L P L
ctgcatgcgatgcatcatggcacaccagaacaggcacagatgatccgtaccgccatcgaa
  L H A M H H G T P E Q A Q M I R T A I E
cagggtaacggtcgccatcttctggaaccggttctggaagcaatgaacgcttgtggatct
  Q G N G R H L L E P V L E A M N A C G S
cttgaatggacgcgtcagcgtgccgaggaagaagcagacaaagccatcgcagcgttacag
  L E W T R Q R A E E E A D K A I A A L Q
gtgctcccggacaccccttggcgagaagcactcatcggcctcgcgcacatcgctgttcaa
  V L P D T P W R E A L I G L A H I A V Q
cgcgatcgttaa
  R D R
Nucleotide sequence (SEQ ID NO: 7) of E. coli ispD gene and
the encoded amino acid sequence (SEQ ID NO: 8)
atggcaaccactcatttggatgtttgcgccgtggttccggcggccggatttggccgtcga
  M A T T H L D V C A V V P A A G F G R R
atgcaaacggaatgtcctaagcaatatctctcaatcggtaatcaaaccattcttgaacac
  M Q T E C P K Q Y L S I G N Q T I L E H
tcggtgcatgcgctgctggcgcatccccgggtgaaacgtgtcgtcattgccataagtcct
  S V H A L L A H P R V K R V V I A I S P
ggcgatagccgttttgcacaacttcctctggcgaatcatccgcaaatcaccgttgtagat
  G D S R F A Q L P L A N H P Q I T V V D
ggcggtgatgagcgtgccgattccgtgctggcaggtctgaaagccgctggcgacgcgcag
  G G D E R A D S V L A G L K A A G D A Q
tgggtattggtgcatgacgccgctcgtccttgtttgcatcaggatgacctcgcgcgattg
  W V L V H D A A R P C L H Q D D L A R L
ttggcgttgagcgaaaccagccgcacgggggggatcctcgccgcaccagtgcgcgatact
  L A L S E T S R T G G I L A A P V R D T
atgaaacgtgccgaaccgggcaaaaatgccattgctcataccgttgatcgcaacggctta
  M K R A E P G K N A I A H T V D R N G L
tggcacgcgctgacgccgcaatttttccctcgtgagctgttacatgactgtctgacgcgc
  W H A L T P Q F F P R E L L H D C L T R
gctctaaatgaaggcgcgactattaccgacgaagcctcggcgctggaatattgcggattc
  A L N E G A T I T D E A S A L E Y C G F
catcctcagttggtcgaaggccgtgcggataacattaaagtcacgcgcccggaagatttg
  H P Q L V E G R A D N I K V T R P E D L
gcactggccgagttttacctcacccgaaccatccatcaggagaatacataa
  A L A E F Y L T R T I H Q E N T -
Nucleotide sequence (SEQ ID NO: 9) of E. coli ispF gene and
the encoded amino acid sequence (SEQ ID NO: 10)
atgcgaattggacacggttttgacgtacatgcctttggcggtgaaggcccaattatcatt
  M R I G H G F D V H A F G G E G P I I I
ggtggcgtacgcattccttacgaaaaaggattgctggcgcattctgatggcgacgtggcg
  G G V R I P Y E K G L L A H S D G D V A
ctccatgcgttgaccgatgcattgcttggcgcggcggcgctgggggatatcggcaagctg
  L H A L T D A L L G A A A L G D I G K L
ttcccggataccgatccggcatttaaaggtgccgatagccgcgagctgctacgcgaagcc
  F P D T D P A F K G A D S R E L L R E A
tggcgtcgtattcaggcgaagggttatacccttggcaacgtcgatgtcactatcatcgct
  W R R I Q A K G Y T L G N V D V T I I A
caggcaccgaagatgttgccgcacattccacaaatgcgcgtgtttattgccgaagatctc
  Q A P K M L P H I P Q M R V F I A E D L
ggctgccatatggatgatgttaacgtgaaagccactactacggaaaaactgggatttacc
  G C H M D D V N V K A T T T E K L G F T
ggacgtggggaagggattgcctgtgaagcggtggcgctactcattaaggcaacaaaatga
  G R G E G I A C E A V A L L I K A T K -
```

Both native DXP genes and their functional variants can be used to construct the exogenous expression cassette mentioned above. Functional variants include degenerative variants of the native DXP genes and nucleotide sequences encoding functional equivalents of the proteins encoded by the native genes. A functional equivalent of a protein refers to a polypeptide that shares at least 80% (e.g., 90%, 95%, or 99%) sequence identity to the protein and has the same bioactivity as the protein.

The percent identity of two amino acid sequences is determined using the algorithm of Karlin and Altschul Proc. Natl. Acad. Sci. USA 87:2264-68, 1990, as modified in Karlin and Altschul Proc. Natl. Acad. Sci. USA 90:5873-77, 1993. Such an algorithm is incorporated into the BLASTN and BLASTX programs (version 2.0) of Altschul et al., J. Mol. Biol. 215:403-10, 1990. BLAST protein searches can be performed with the BLASTX program, score=50, wordlength=3 to obtain amino acid sequences homologous to the protein molecules of the invention. Where gaps exist between two sequences, Gapped BLAST can be utilized as described in Altschul et al., Nucleic Acids Res. 25:3389-3402, 1997. When utilizing BLAST and Gapped BLAST programs, the default parameters of the respective programs (e.g., BLASTX and BLASTN) can be used.

The genetically modified E. coli strain described above can further include another expression cassette that overly expresses a txs gene (encoding taxadiene synthase) and a crtE gene (encoding geranylgeranyl pyrophosphate synthase). txs and crtE genes have also been identified in various species, including Synechococcus sp., T. baccata, A. brandis, and P. abies. Table 2 below lists a number of txs and crtE genes, the corresponding species from which they derive, and the GenBank accession numbers of their encoded proteins:

TABLE 2
Exemplary crtE and txs genes

| Gene | Species | | | |
|---|---|---|---|---|
| Txs | T. baccata | T. wallichiana | A. grandis | P. abies |
| | AAR02861 | AAY16197 | AAC24192 | AAS47689 |
| | GI: 37789216 | GI: 62825333 | GI: 3252840 | GI: 44804486 |
| | (Oct. 26, 2003) | (Apr. 10, 2006) | (Jun. 24, 1998) | (Aug. 29, |
| | | | | 2004) |
| crtE | F. vulneris | P. | E. sakazakii | A. thaliana |
| | P22873 | agglomerans | CAL34117 | BAA19583 |
| | GI: 117508 | AAA24819 | GI: 112702897 | GI: 1944371 |
| | (Sep. 2, 2008) | GI: 148400 | (Aug. 17, | (Feb. 14, 2004) |
| | | (Apr. 11, 2001) | 2006) | |

The sequences of the P. agglomerans crtE gene and T. baccata txs gene and their encoded amino acid sequences are shown below:

```
Nucleotide sequence (SEQ ID NO: 11) of P. agglomerans crtE
gene and the encoded amino acid sequence (SEQ ID NO: 12)
atggtgagtggcagtaaagcgggcgtttcgcctcatcgcgaaatagaagtaatgagacaa
  M V S G S K A G V S P H R E I E V M R Q
tccattgacgatcacctggctggcctgttacctgaaaccgacagccaggatatcgtcagc
  S I D D H L A G L L P E T D S Q D I V S
cttgcgatgcgtgaaggcgtcatggcacccggtaaacggatccgtccgctgctgatgctg
  L A M R E G V M A P G K R I R P L L M L
ctggccgcccgcgacctccgctaccagggcagtatgcctacgctgctcgatctcgcctgc
  L A A R D L R Y Q G S M P T L L D L A C
gccgttgaactgacccataccgcgtcgctgatgctcgacgacatgccctgcatggacaac
  A V E L T H T A S L M L D D M P C M D N
gccgagctgcgccgcggtcagcccactacccacaaaaaatttggtgagagcgtggcgatc
  A E L R R G Q P T T H K K F G E S V A I
cttgcctccgttgggctgctctctaaagcctttggtctgatcgccgccaccggcgatctg
  L A S V G L L S K A F G L I A A T G D L
ccgggggagaggcgtgcccaggcggtcaacgagctctctaccgccgtgggcgtgcagggc
  P G E R R A Q A V N E L S T A V G V Q G
ctggtactggggcagtttcgcgatcttaacgatgccgccctcgaccgtacccctgacgct
  L V L G Q F R D L N D A A L D R T P D A
atcctcagcaccaaccacctcaagaccggcattctgttcagcgcgatgctgcagatcgtc
  I L S T N H L K T G I L F S A M L Q I V
gccattgcttccgcctcgtcgccgagcacgcgagagacgctgcacgccttcgccctcgac
  A I A S A S S P S T R E T L H A F A L D
ttcggccaggcgtttcaactgctggacgatctgcgtgacgatcacccggaaaccggtaaa
  F G Q A F Q L L D D L R D D H P E T G K
gatcgcaataaggacgcgggaaaatcgacgctggtcaaccggctgggcgcagacgcggcc
  D R N K D A G K S T L V N R L G A D A A
cggcaaaagctgcgcgagcatattgattccgccgacaaacacctcacttttgcctgtccg
  R Q K L R E H I D S A D K H L T F A C P
cagggcggcgccatccgacagtttatgcatctgtggtttggccatcaccttgccgactgg
  Q G G A I R Q F M H L W F G H H L A D W
tcaccggtcatgaaaatcgcctga
  S P V M K I A -
Nucleotide sequence (SEQ ID NO: 13) of T. baccata txs gene
and the encoded amino acid sequence (SEQ ID NO: 14)
atgagcagtagcactggcactagcaaggtggtttccgagacttccagtaccattgtggat
  M S S S T G T S K V V S E T S S T I V D
gatatccctcgactctccgccaattatcatggcgatctgtggcaccacaatgttatacaa
  D I P R L S A N Y H G D L W H H N V I Q
actctggagacaccatttcgtgagagttctactttccaagaacgggcagacgagctggtt
  T L E T P F R E S S T F Q E R A D E L V
gtgaaaattaaagatatgttcaatgcgctcggagacggagatatcagtccgtctgcatac
  V K I K D M F N A L G D G D I S P S A Y
gacactgcgtgggtggcgagggtggcgaccgtttcctctgatggatctgagaagccacgg
  D T A W V A R V A T V S S D G S E K P R
tttcctcaggccctcaactgggttttaaacaaccagctccaagatggatcatggggtatc
  F P Q A L N W V L N N Q L Q D G S W G I
gaatcgcactttagtttatgcgatcgattgcttaacacggtcaattctgttatcgccctc
  E S H F S L C D R L L N T V N S V I A L
tcggtttggaaaacagggcacagccaagtagaacaaggtactgagtttattgcagagaat
  S V W K T G H S Q V E Q G T E F I A E N
ctaagattactcaatgaggaagatgagttgtccccggatttcgaaataatctttcctgct
  L R L L N E E D E L S P D F E I I F P A
ctgctgcaaaaggcaaaagcgttggggatcaatcttccttacgatcttccatttatcaaa
  L L Q K A K A L G I N L P Y D L P F I K
tctttgtcgacaacacgggaagccaggcttacagatgtttctgcggcagcagacaatatt
  S L S T T R E A R L T D V S A A A D N I
ccagccaacatgttgaatgcgttggagggtctggaggaagttattgattggaacaagatt
  P A N M L N A L E G L E E V I D W N K I
atgaggtttcaaagtaaagatggatctttcctgagctcccctgcctccactgcctgtgta
  M R F Q S K D G S F L S S P A S T A C V
ctgatgaatacaggggacgaaaaatgtttcactcttctcaacaatctgctggacaaattc
  L M N T G D E K C F T L L N N L L D K F
ggcggctgcgtgccctgtatgtattccatcgatctgctggaacgcctttcgctggttgat
  G G C V P C M Y S I D L L E R L S L V D
aacattgagcatctcggaatcggtcgccatttcaaacaagaaatcaaagtagctcttgat
  N I E H L G I G R H F K Q E I K V A L D
tatgtctacagacattggagtgaaaggggcatcggttggggcagagacagccttgttcca
  Y V Y R H W S E R G I G W G R D S L V P
gatctcaacacaacagccctcggcctgcgaactcttcgcacgcacggatacgatgtttct
  D L N T T A L G L R T L R T H G Y D V S
tcagatgttttgaataatttcaaagatgaaaacgggcggttcttctcctctgcgggccaa
  S D V L N N F K D E N G R F F S S A G Q
acccatgtcgaattgagaagcgtggtgaatcttttcagagcttccgaccttgcatttcct
  T H V E L R S V V N L F R A S D L A F P
gacgaaggagctatggacgatgctagaaaatttgcagaaccatatcttagagacgcactt
  D E G A M D D A R K F A E P Y L R D A L
gcaacgaaaatctcaaccaatacaaaactatacaaagagattgagtacgtggtggagtac
  A T K I S T N T K L Y K E I E Y V V E Y
ccttggcacatgagtatcccacgcctagaagctagaagttatattgattcgtatgacgac
  P W H M S I P R L E A R S Y I D S Y D D
gattatgtatggcagaggaagactctatacagaatgccatctttgagtaattcaaaatgt
  D Y V W Q R K T L Y R M P S L S N S K C
ttagaattggcaaaattggacttcaatatcgtacaatctttgcatcaagaggagttgaag
  L E L A K L D F N I V Q S L H Q E E L K
cttctaacaagatggtggaaggaatccggcatggcagatataaatttcactcgacaccga
  L L T R W W K E S G M A D I N F T R H R
gtggcggaggtttatttttcatcagctacatttgaacctgaatattctgccaccagaatt
  V A E V Y F S S A T F E P E Y S A T R I
gccttcacaaaaattggttgtttacaagtcctttttgatgatatggctgacatctttgca
  A F T K I G C L Q V L F D D M A D I F A
acactagatgaattgaaaagtttcactgagggagtaaagagatgggatacatctttgcta
  T L D E L K S F T E G V K R W D T S L L
catgagattccagagtgtatgcaaacttgctttaaagtttggttcaaattaatggaagaa
  H E I P E C M Q T C F K V W F K L M E E
gtaaataatgatgtggttaaggtacaaggacgtgacatgctcgctcacataagaaaacct
  V N N D V V K V Q G R D M L A H I R K P
tgggagttgtacttcaattgttacgtacaagaaagggagtggcttgaagctgggtatata
  W E L Y F N C Y V Q E R E W L E A G Y I
ccaacttttgaagagtacttaaagacttatgctatatcagtaggccttggaccgtgtacc
  P T F E E Y L K T Y A I S V G L G P C T
ctacaaccaatactactgatgggtgagcttgtgaaagatgatgttgttgagaaagtgcac
  L Q P I L L M G E L V K D D V V E K V H
tatccctcaaatatgtttgagcttgtatccttgagctggcgactaacaaacgacaccaaa
  Y P S N M F E L V S L S W R L T N D T K
acatatcaggctgaaaaggctcgaggacaacaagcctcaggcatagcatgctatatgaag
  T Y Q A E K A R G Q Q A S G I A C Y M K
gataatccaggagcaactgaggaagatgccatcaagcacatatgtcgtgttgttgaccgg
  D N P G A T E E D A I K H I C R V V D R
gccttgaaagaagcaagctttgaatatttcaaaccatccaatgatatcccaatgggttgc
  A L K E A S F E Y F K P S N D I P M G C
aagtcctttatttttaaccttagattgtgtgtccaaatattttacaagtttatagatggg
  K S F I F N L R L C V Q I F Y K F I D G
tacggaatcgccaatgaggagattaaggattatataagaaaagtttatattgatccaatt
  Y G I A N E E I K D Y I R K V Y I D P I
caagtatga
  Q V -
```

Both native crtE and txs genes and their functional variants can be used to construct the other expression cassette, which can be extra-chromosomal. When a non-E. coli crtE or txs gene is used, it is preferable to optimize the sequence in accordance with E. coli codon preferences to improve the gene's expression in E. coli. Below are the nucleotide sequences of thus optimized crtE gene and txs genes:

```
Nucleotide sequence (SEQ ID NO: 15) of T. cuspidata crtE gene
optimized for codon usage in E. coli and the encoded amino acid
sequence (SEQ ID NO: 16)
atgttcgacttcaacgagtacatgaaatcgaaagcagttgcagttgatgctgcgcttgac
  M F D F N E Y M K S K A V A V D A A L D
aaagcgattccgctggaataccctgaaaagattcacgaatcgatgcgttatagtctgctg
  K A I P L E Y P E K I H E S M R Y S L L
gctggtggcaaacgcgtgcgcccagctctttgcattgcggcatgtgagctggtaggcggt
  A G G K R V R P A L C I A A C E L V G G
tcccaggatctggctatgccaacggcgtgcgcaatggaaatgatccatacaatgtccctg
  S Q D L A M P T A C A M E M I H T M S L
atccacgatgatctgccgtgtatggataatgatgacttccgccgtggaaaaccgactaac
  I H D D L P C M D N D D F R R G K P T N
cataaagtatttggcgaggacactgcagtgttggcaggagacgccctgttgagctttgcc
  H K V F G E D T A V L A G D A L L S F A
tttgaacatattgccgtcgcgacctcaaaaacagttccttctgatcgtaccctgcgcgtc
  F E H I A V A T S K T V P S D R T L R V
atcagtgagttaggtaagaccattggcagccaggggctggtaggcggccaggtcgtggat
  I S E L G K T I G S Q G L V G G Q V V D
atcacgtctgaaggtgacgcgaatgtggatcttaagaccttagagtggattcacatccat
  I T S E G D A N V D L K T L E W I H I H
aaaacggccgtgctgctggaatgctcggttgtgtccggtgggatcctggggggcgccact
  K T A V L L E C S V V S G G I L G G A T
gaggacgaaatcgcccgtattcgccgttatgcacggtgtgtgggcctcttgtttcaagtc
  E D E I A R I R R Y A R C V G L L F Q V
gtggatgatattctggatgtgacgaaatctagtgaggagctcggtaaaaccgcgggcaag
  V D D I L D V T K S S E E L G K T A G K
gatctcctgaccgacaaggcgacgtacccgaaactgatgggtttggaaaaggctaaggag
  D L L T D K A T Y P K L M G L E K A K E
tttgctgccgaattagcgaccagagccaaagaagaactctcttctttcgaccagatcaag
  F A A E L A T R A K E E L S S F D Q I K
gcagcgccccttttagggctggcggattatattgcctttcgtcaaaactaa
  A A P L L G L A D Y I A F R Q N -
Nucleotide sequence (SEQ ID NO: 17) of T. baccata txs gene optimized
for codon usage in E. coli
catatgatgtctagctctacgggtacgtctaaagtcgtgagtgaaacctcatcgacgatcgtggacgata
ttccacgcttgtcggcgaactatcatggagatctgtggcatcataacgtcattcagacattggaaacccc
gtttcgcgaaagtagcacctaccaggaacgggcagatgaattagtcgtgaaaatcaaagatatgtttaat
gcattaggagatggagacatctcgcccagcgcatatgatacggcgtgggtggctcggttggccacgatta
gctccgatggcagtgaaaagccgcgtttcccgcaggcgctgaactgggtgtttaataatcaattgcagga
tggcagctggggcattgaatctcactttagcctctgtgaccggttactcaacacgacaaactccgtaatt
gcgttgtcagtttggaaaacgggccatagccaggttcaacagggcgcggaatttatcgctgaaaatctgc
gcctgctgaacgaggaggacgaactgtcacccgattttcagattatttttccggctttactccagaaagc
caaagccttaggcatcaacctgccatatgatctgccgttcatcaagtatctgtctactacccgcgaagcc
cgtctcactgacgtctctgcggcggcggacaatattccagcgaacatgctgaacgcactggaagggctgg
aagaggttatcgactggaataaaatcatgcgcttccaaagcaaggacggtagcttcttaagcagcccagc
atctactgcttgtgttctgatgaataccggagacgaaaagtgctttacgtttctgaacaatctgctggac
aaatttgggggttgtgttccttgtatgtattccattgatctgttggaacgtctgtcgctggtcgataaca
ttgaacacttaggtatcggccgccacttcaaacaagaaatcaagggggcgttggattatgtataccgtca
ttggagcgagcgtggtattggttgggggcgcgatagcttggtacctgatctgaacaccactgctttggga
ctgcgcactcttcgtatgcacggatacaacgttagttccgatgtcctcaataatttcaaggacgagaacg
gccgttttttcagctcggccggtcagacgcatgttgaactgcggtccgtagtcaatctctttcgcgctag
tgatctggccttccccgacgagcgcgctatggacgatgcacggaagtttgccgagccgtatctccgcgaa
gccctggccaccaaaatttcaaccaacaccaagcttttcaaagaaattgagtatgtagtagagtatccgt
ggcatatgtctattccgcgcctggaagcccgctcgtatatcgattcttacgatgacaattatgtgtggca
acgcaaaacactgtaccgtatgcccagcctgtcaaatagtaagtgtctggagctggcgaaactggatttc
aacattgtgcaatccctgcaccaagaagagctgaaattactgactcgctggtggaaggaatccggcatgg
cagacatcaattttacgcgtcaccgtgttgcagaggtgtacttctcctcggcgacctttgagccggagta
ttcggccacacgtattgcatttaccaagattggctgccttcaggtgctttttgacgatatggcggatatt
tttgcgacacttgatgagcttaaatcatttaccgaaggcgtgaagcgttgggatacctctctgttgcatg
aaatccccgaatgtatgcagacctgcttcaaagtttggttcaaactgatggaagaagtgaacaacgacgt
cgtgaaagttcagggtcgtgatatgttagcacacatccgcaagccgtgggaactctatttcaattgctat
gtgcaggagcgtgaatggttagaagcgggctacattcctaccttcgaagagtacttaaaaacctatgcca
tttccgtcggtttaggcccgtgcactctgcagcctatcttgctgatgggtgagctggtaaaggatgatgt
ggtggaaaaagttcactacccgtcgaatatgtttgaactggtaagtctgagttggcgtctgacaaacgac
accaaaacgtaccaggcagaaaaggcacgtgggcaacaggcaagcggtatcgcgtgttatatgaaggata
atccgggcgctactgaggaagatgccattaagcatatctgccgtgttgtggatcgcgctcttaaagaagc
gtcattcgaatattttaaacctagtaatgatattccgatgggttgtaagtcattcattttcaatcttcgc
ctgtgcgtgcaaattttttacaaatttattgacggctacggaatcgccaacgaagaaatcaaagactata
ttcgtaaagtttacatcgatccaatccaggtctaa
```

Either the exogenous expression cassette alone or together with the other expression cassette can be introduced into a host E. coli strain, e.g., JM109, BL21(DE3), DH5α, and MC1061, via conventional methods to produce a genetically modified E. coli strain. In one example, the host E. coli has a disrupted gdhA, aceE, or fdhF gene, which does not express a functional protein. See Jin et al., Metab. Eng. 9:337-347, 2007. Preferably, the exogenous expression cassette is integrated into the chromosome of the host E. coli, e.g., by homologous recombination (see Yuan et al., Metab. Eng., 8:79-90, 2006) and the other expression cassette is extra-chromosomal.

Any of the genetically modified E. coli strains, containing either the exogenous expression cassette alone or together with the other expression cassette, can be cultured under conditions suitable for over-expression of the genes included in the cassettes. For example, the E. coli strain can be cultivated in a culture medium containing an elevated level of glycerol (e.g., 10-100 g/L), an elevated level of yeast extract (e.g., 5-25 g/L), or in the presence of an anti-foam agent, e.g., Antifoam B. Any yeast extract commonly used for preparing bacterial culture media can be used herein. These yeast extracts can be purchased from a reputable vendor, e.g., Difco, Invitrogen, Fisher Scientific, and Sigma.

The screening assay noted above can be performed as follows. Microbial DNAs are obtained following methods well known in the art (see, e.g., MacNeil et al., J Molec Microbio & Biotech, 3:301-308, 2001; Rondon et al., Proc Natl Acad Sci, USA 96:6451-6455, 1999; Rondon et al., Appl Environ Microbiol., 66:2541-5247, 2000; Somerville et al., Appl Environ Microbiol, 55:548-554, 1989; Zhou et al., Appl Environ Microbiol, 62:316-322, 1996). The DNAs are then packaged into suitable expression vectors in mass using conventional recombinant technology to generate a library containing various expression plasmids each for expression of a microbial DNA. The expression plasmids, capable of expressing the microbial DNAs in E. coli, are introduced into cells of the genetically modified E. coli strain using techniques well known in the art (e.g., chemical transformation or electroporation). The transformant clones are cultured either separately or together in conditions suitable for overexpression of the expression cassette's genes present in the modified E. coli cells, as described above.

The microbial DNA-carrying transformants that synthesize therapeutic terpenoid-based compounds can be identified by methods well known in the art.

Described below is an exemplary method of identifying a transformant secreting a terpenoid-based antibiotic. The transformant is first grown on a solid culture medium to form a colony. The colony is then placed on or overlaid with a soft agar containing a confluent lawn of a tester microorganism e.g., B. subtilis or S. cerevisiae (see Rodriguez-Pena et al., J. Biotech. 133:311-7, 2008). If the transformant secretes an antibiotic compound, a zone of inhibition is formed surrounding the transformant colony. Secretion of an antibiotic indicates that the transformant carries a microbial DNA that encodes an enzyme or enzyme(s) involved in the synthesis of the antibiotic.

Another example of identifying a transformant that secretes a terpenoid-based antibiotic follows. The transformant is cultivated in a liquid culture medium to allow secretion of a terpenoid-based antibiotic, if any, into the medium. The culturing medium is then collected by centrifugation and directly applied to a tester microorganism by adding the medium to a liquid culture of the tester microorganism or overlaying the medium onto a confluent lawn of the tester microorganism. Presence or absence of the antibiotic in the medium can be determined based on growth inhibition of the tester microorganism.

To identify a microbial DNA-transformant that secrets an anti-cancer terpenoid-based compound, the culturing medium mentioned immediately above is applied to cancer cells. A cytotoxic effect on the cancer cells can be determined by conventional procedures.

If desired, all of the methods described above is performed in a high throughput manner, in which a plurality of microbial DNAs are tested simultaneously. A pool of microbial DNA-transformants is tested to obtain subpopulations of the transformants that are enriched for those that produce antibiotics or anti-cancer compounds. Sequential screening of the subpopulations narrows down candidates and ultimately identifies individual transformants that carry microbial DNAs of interest.

When a transformant is found to secrete a therapeutic terpenoid-based compound, its microbial DNA is isolated, characterized, and expressed to produce the encoding enzyme(s). Such an enzyme(s) can be used to prepare the terpenoid-based compound. Also, the transformant can be cultured in large scale to produce a culturing medium containing the terpenoid-based compound. The compound can be purified for medical or other uses.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Construction of Genetically Modified E. coli Strains that Produce High Levels of Enzymes in the Non-Mevalonate 1-Deoxy-D-xylulose-5-Phosphate Synthetic Pathway

E. coli strains MC1061, JM109(DE3), and BL21(DE3) were used as original hosts for generating the genetically modified E. coli strains YW140, YWGAF, YW22, and YW23. Strains YW140 and YWGAF were constructed as described in Yuan et al., Metab. Eng. 8:79-90, 2006 and Jin et al., Metab. Eng., 9:337-347, 2007. An expression cassette containing, from 5′ to 3′, a T7 promoter, dxs, an RBS, idi, an RBS, ispB, an RBS, ispDF, and a T7 terminator (i.e., T7-dxs-RBS-idi-RBS-ispB-RBS-ispDF-Term) was assembled by conventional recombinant technology and cloned into pET21c to produce an expression plasmid. See Pfeifer et al., Science 291:1790-1792, 2001. The coding sequences of these (E. coli) genes are provide above. The resultant plasmid was then integrated into the chromosome of JM109(DE3) and BL21(DE3) following the method described in Chen et al., Chinese Journal of Biotechnology, 21:192-197, 2005 and Wang et al., Metab Eng 10:33-38, 2008, to produce strains YW22 and YW23. The major features of E. coli strains YW140, YWGAF, YW22, and YW23 are summarized in Table 3 below:

TABLE 3
Genetically modified E. coli strains used in Example 1

| Strain | Description |
|---|---|
| YW22 | JM109(DE3) with integrated cassette (T7prom-dxs- |
| | RBS-idi-RBS-ispB-RBS-ispDF-Term) into araA |
| | location |
| YW23 | BL21(DE3) with integrated cassette (T7prom-dxs-RBS- |
| | idi-RBS-ispB-RBS-ispDF-Term) into araA location |
| YWS140 | MC1061 with dxs, idi, and ispDF genes over-expressed |
| | through T5 promoter replacement |
| YWGAF | YWS140 with deletions to gdhA, aceE, fdhF genes |

Abbreviations:
prom—promoter,
term—terminator.

### Example 2

### Screening for Microbial DNAs Encoding Enzymes Involved in Terpenoid-Based Anti-Cancer Compounds Using Genetically Modified E. coli Strains

Microbial DNAs are collected from a soil sample as follows. Soil containing various microbes is sieved to remove particles larger than 1 cm. 5 g sieved soil is placed into 13.5 ml of a DNA extraction solution containing 100 mM Tris-HCl (pH 8.0), 100 mM sodium EDTA (pH 8.0), 100 mM sodium phosphate (pH 8.0), 1.5 M NaCl, 1% Hexadecylmethylammonium bromide, and 100 μl of proteinase K (concentration: 10 mg/ml) to form a mixture. After being shaken for 30 min at 37° C., the mixture is combined with 1.5 ml of 20% SDS and then incubated at 65° C. for 2 hours. The supernatant is collected after centrifugation and mixed with an equal volume of phenol-chloroform isoamyl alcohol (25:24:1, v/v/v). The aqueous phase is recovered and mixed with 0.6 volume of isopropanol for an hour to precipitate the nucleic acid contained therein. The pellet of crude nucleic acid is obtained by centrifugation at room temperature, washed with cold 70% ethanol, and resuspended in sterile deionized water to give a final volume of 500 ml. Alternately, microbial DNAs can be prepared using commercially available DNA prep kits, e.g., from Qiagen. The resultant DNAs are stored at −20° C. until use.

Microbial DNAs are isolated from an aquatic sample as follows. Water from a natural source is vacuum filtered through sterile 0.22 μm pore filter paper, which traps aquatic microbes. The filter paper is then transferred to a 50 ml conical tube. The tube is filled with sterile 100 mM Tris-HCl (pH 8.0) and vortexed to separate the microbes from the filter paper. After centrifugation, the pelleted microbes are resuspended in the above-mentioned DNA extraction solution to extract microbial DNA, following the method described above.

The microbial DNAs prepared as described above are electrophoresed in a low-melting-temperature agarose gel. After electrophoresis at 100 V for 1 h, the DNA-containing region is cut from the gel and the DNAs are electroeluted into dialysis membranes and dialyzed overnight against a Tris-EDTA buffer. The DNAs are then digested with HindIII, inserted into the expression vector pBeloBAC11, and the resultant plasmids are introduced into a suitable E. coli host according to Rondon et al., Proc. Natl. Acad. Sci. USA 96:6451-6455, 1999. Transformants are collected in mass and the expression plasmids contained in them are extracted using BAC-plasmid prepping techniques to generate a library of microbial DNA expression plasmids. See bacpac.chori.org/bacpacmini.htm. Alternately, BAC DNA can be prepared using commercially available BAC DNA prep kits, e.g., from Qiagen.

This library is introduced into the genetically modified YW22, YW23, YWS140, or YWGAF host cells via electroporation. The resultant set of microbial DNA-transformed cells is diluted to a concentration that allows single colony resolution from an overnight growth on standard LB agar Petri dishes. A number of colonies are picked, each being placed in a well of a 48-well plate containing a culturing medium suitable for overexpressing the DXP genes carried by the host cells. Isopropyl β-D-1-thiogalactopyranoside (IPTG) is then added (final concentration 100 μM) to induce microbial DNA expression. The plate is incubated for 48 hours at 30° C. and the culturing medium is collected.

The culturing medium is then examined for cytotoxic effect on cancer cells in vitro as follows. 4T1 tumor cells and D2.OR non-tumor cells (Tao et al., Int J Oncology, 19:1333-1339, 2001) are respectively transfected with pGL4 expression plasmids (Promega) containing the DNA coding for firefly luciferase and renilla luciferase (as described in Li et al., Oncogene 23:5739-5747, 2004). Co-cultures of firefly luciferase-expressing 4T1 cells and renilla luciferase-expressing D2.OR cells are grown in Dulbecco's Modified Eagle's Medium in 96-well plates until cells are nearly confluent. A well of the co-culture is then incubated in a fresh Dulbecco's Modified Eagle's Medium containing the culturing medium 10% (v/v) to be examined. After a 72 hour incubation, the numbers of 4T1 and D2.OR cells in the well is determined by biophotonic imaging following sequential addition of luciferin (for firefly luciferase) and coelenterazine (for renilla luciferase).

A transformant culturing medium that selectively kills 4T1 tumor cells is traced back to the transformant clone. The microbial DNA carried by that clone, encoding an enzyme(s) for synthesizing an anti-cancer terpenoid-based compound, is isolated and analyzed using standard molecular biology techniques.

The anti-cancer terpenoid-based compound contained in the culturing medium is identified as follows. The culturing medium is subjected to size exclusion and reverse phase preparatory high performance liquid chromatographies (HPLC) to produce a series of fractions, each containing a terpenoid-based compound. These fractions are then tested for their cytotoxic activity following the method described above to identify those that exhibit anti-cancer activity. The fractions that show cytotoxicity are then tested against a panel of human tumor and normal cell lines to assess the specificity and generality of the terpenoid-based compounds contained therein for killing or inhibiting growth of tumor cells along with minimal effects on normal cells.

Finally, the anti-cancer activity of the identified terpenoid-based compounds are confirmed in vivo. Luciferase-expressing 4T1 tumor cells (10⁶) are injected into 4-6 week old female BALB/c mice. Tumors are allowed to grow for 3 weeks, when they will be approximately 0.5 cm in diameter. One of the anti-cancer terpenoid-based compounds are administered to the tumor-bearing mice by intraperitoneal injection every two days, at an acceptable dose, for a period of 6 weeks. The mice are imaged on a weekly basis to quantify growth or regression of the tumors. Postmortem analyses are conducted at the end of the 6 weeks to determine the morphological and molecular effects of the anti-cancer compounds on tumor cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for determining whether a microbial DNA encodes an enzyme or enzymes for synthesis of a terpenoid-based antibiotic, said method comprising
transforming a genetically modified E. coli strain with an expression plasmid carrying a microbial DNA to obtain a transformant, wherein the E. coli strain contains an exogenous expression cassette including an E. coli promoter, a dxs gene, and an idi gene, the E. coli promoter being in operative linkage with the dxs and idi genes,
contacting a microorganism suspected of being sensitive to a terpenoid-based antibiotic with substances released from the transformant, and
determining whether or not the substances inhibit growth of the microorganism,
wherein detection of growth inhibition indicates that the microbial DNA encodes an enzyme or enzymes for synthesis of a terpenoid-based antibiotic.

2. The method of claim 1, wherein the exogenous expression cassette is integrated into the chromosome of the E. coli strain.

3. The method of claim 2, wherein the exogenous expression cassette is integrated into the E. coli chromosome at its araA locus.

4. The method of claim 1, wherein the gdhA, aceA, and fdhF genes are non-functional in the E. coli strain.

5. The method of claim 1, wherein the exogenous expression cassette further includes a ribosomal binding site adjacent to the 5′ end of each of the genes contained therein.

6. The method of claim 1, wherein the exogenous expression cassette further includes an ispB gene, an ispD gene, or an ispF gene, the E. coli promoter being in operative linkage with the ispB, ispD, or ispF genes.

7. The method of claim 1, wherein the exogenous expression cassette further includes an ispB gene, an ispD gene, and an ispF gene, the E. coli promoter being in operative linkage with the ispB, ispD, and ispF genes.

8. The method of claim 7, wherein the exogenous expression cassette is integrated into the chromosome of the E. coli strain at its araA locus.

9. The method of claim 8, wherein the gdhA, aceA, and fdhF genes are non-functional in the E. coli strain.

10. The method of claim 1, wherein said method is a high throughput screening assay in which a plurality of microbial DNAs is tested.

11. The method of claim 1, wherein the contacting step is performed by cultivating the transformant in a culturing medium to allow expression of the microbial DNA, collecting the culturing medium after the cultivation, and contacting the culturing medium with the microorganism.

12. The method of claim 1, wherein the contacting step is performed by cultivating the transformant to form a colony and contacting the colony with a lawn of the microorganism.

13. A method for determining whether a microbial DNA encodes an enzyme or enzymes for synthesis of a terpenoid-based anti-cancer compound, said method comprising
transforming a genetically modified E. coli strain with an expression plasmid carrying a microbial DNA to obtain a transformant, wherein the E. coli strain contains an exogenous expression cassette including an E. coli promoter, a dxs gene, and an idi gene, the E. coli promoter being in operative linkage with the dxs and idi genes,
cultivating the transformant in a culturing medium to allow expression of the microbial DNA,
collecting the culturing medium after the cultivation,
contacting the culturing medium with cancer cells, and
determining whether or not the culturing medium exerts cytotoxic effect on the cancer cells,
wherein detection of cytotoxic effect indicates that the microbial DNA encodes an enzyme or enzymes for synthesis of a terpenoid-based anti-cancer compound.

14. The method of claim 13, wherein the exogenous expression cassette is integrated into the chromosome of the E. coli strain.

15. The method of claim 14, wherein the exogenous expression cassette is integrated into the E. coli chromosome at its araA locus.

16. The method of claim 13, wherein the gdhA, aceA, and fdhF genes are non-functional in the E. coli strain.

17. The method of claim 13, wherein the exogenous expression cassette further includes a ribosomal binding site adjacent to the 5′ end of each of the genes contained therein.

18. The method of claim 13, wherein the exogenous expression cassette further includes an ispB gene, an ispD gene, or an ispF gene, the E. coli promoter being in operative linkage with the ispB, ispD, or ispF genes.

19. The method of claim 13, wherein the exogenous expression cassette further includes an ispB gene, an ispD gene, and an ispF gene, the E. coli promoter being in operative linkage with the ispB, ispD, and ispF genes.

20. The method of claim 19, wherein the exogenous expression cassette is integrated into the chromosome of the E. coli strain at its araA locus.

21. The method of claim 20, wherein the gdhA, aceA, and fdhF genes are non-functional in the E. coli strain.

22. The method of claim 13, wherein said method is a high throughput screening assay in which a plurality of microbial DNAs is tested.
